(19) United States
(12) Patent Application Publication
(10) Pub. No.: US 2002/0165118 A1
(43) Pub. Date: Nov. 7, 2002

(54) Hypotension

(76) Inventors: Walter J. Koch, Durham, NC (US); Andrea D. Eckhart, Durham, NC (US)

Correspondence Address: NIXON & VANDERHYE P.C., 8th Floor, 1100 North Glebe Road, Arlington, VA, 22201, US

(21) Appl. No.: 09/984,867

(22) Filed: Oct. 31, 2001

Related U.S. Application Data
(60) Provisional application No. 60/244,210, filed on Oct. 31, 2000.

(51) Int. Cl. A01K 67/027; A61K 31/00

(52) U.S. Cl. 514/1; 800/18; 800/3

(57) ABSTRACT

The present invention relates, in general, to hypotension and, in particular, to a novel therapeutic target for lowering blood pressure.

This application claims priority from Provisional Application No. 60/244,210, filed Oct. 31, 2000, the entire content of which is incorporated herein by reference.

### TECHNICAL FIELD

The present invention relates, in general, to hypotension and, in particular, to a novel therapeutic target for lowering blood pressure.

### BACKGROUND

High blood pressure or hypertension is a prevalent disease in the United States and is a leading cause of morbidity and mortality. Understanding the mechanisms that lead to hypertension is critical to improve existing health care for this disorder.

A functional abnormality leading to hypertension can reflect an imbalance between G protein-coupled receptor (GPCR) mediated vasoconstriction and vasodialation. GPCR signaling is mediated via heterotrimeric G proteins which, following receptor activation, dissociate and release activated Gα and Gβγ subunits, both of which are capable of molecular signaling (Post et al, FASEB J. 10:741-749 (1996)).

One G protein family member, Gq, is activated by several GPCRs mediating vasoconstriction and its activation can dramatically increase systemic vascular resistance (SVR), mean arterial pressure (MAP) as well as myocardial and vascular hypertrophy. Vasoconstriction can occur following the stimulation of either α₁-adrenergic receptors (ARs) or angiotensin II receptors which activate Gq leading to the accumulation of diacylglycerol and inositol 1,4,5-trisphosphate (IP₃), ultimately leading to the activation of protein kinase C (PKC).

PKC activity and calcium signaling results in constriction of blood vessels (Garrison, Hypertension Primer 2^{nd} Edition, eds Izzo and Black, American Heart Association, pgs. 63-65 (1999)). Vascular hypertrophy occurs during chronic hypertension and also contributes to the elevation of SVR. Activation of Gq through α₁-AR and angiotensin II receptors has also been associated with hypertrophy of VSM in vitro and in vivo in animal models and humans (Duff et al, Cardiovasc. Res. 30:511-517 (1995); Rizzoni et al, Hypertension 35:931-935 (2000); Xin et al, Mol. Pharmacol. 51:764-765 (1997)). Therefore, regulation of signaling through GPCRs is not only critical to maintain tone, but it also appears to be essential to direct growth of the vascular system.

Interestingly, several drugs currently on the market for treating hypertension are GPCR antagonists that block the activation of Gq-coupled receptors such as α₁-ARs, angiotensin II receptors and endothelin I receptors. Thus, multiple input from GPCR's coupled to Gq can lead to increased SVR and lead to hypertension. These drugs targeting a specific Gq-coupled receptor can be effective anti-hypertensive agents, however, other Gq-coupled agonists still are able to exert their influence and can still contribute to a hypertensive state. In fact, several patients take multiple drugs to control their hypertension.

The present invention is based on studies designed to examine the impact of inhibiting all vascular Gq signaling in vivo to determine its contribution to regulation of vascular tone and hypertrophy, especially in the setting of hypertension.

### SUMMARY OF THE INVENTION

The present invention relates to a novel therapeutic target for lowering blood pressure.

Objects and advantages of the present invention will be clear from the description that follows.

### BRIEF DESCRIPTION OF THE DRAWINGS

FIG. 1: Schematic of the transgene used to generate SM22α-GqI transgenic mice. Arrows indicate location of annealing for primers used for PCR.

FIG. 2: Conscious Mean Arterial Pressure (MAP) in NLC versus GRK2 overexpressing (SM22BK10) and GqI expressing (SM22Gq1) mice. Blood pressure measurements were made using a left carotid catheter that was tunneled subcutaneously through to the back. At the time of blood pressure measurement, the mice were conscious and moving freely in their cages. The investigator was blinded to the genotype of the animals. n-size is as noted. *p<0.05 versus NLC, unpaired, two-tailed Student's t-test.

FIGS. 3A and 3B: The presence of an inhibitor of Gq signaling (GqI) in vascular smooth muscle is sufficient to decrease high blood pressure induced by the 2 kidney/1 clip model of hypertension.

### DETAILED DESCRIPTION OF THE INVENTION

There are multiple agents that signal via the G protein, Gq, leading to an increase in SVR and high blood pressure. The present invention results from studies designed to test the hypothesis that this molecule is a common pathological link to this disease and that by targeting all signals through Gq, a more effective anti-hypertensive drug can be developed. Those studies include the development of a specific Gq-inhibitor and targeting of it to the vascular smooth muscle (VSM) of transgenic mice.

It has been previously shown that a peptide comprised of the last 50 amino acids of murine Gαq (Gαq³⁰⁵⁻³⁵⁹ or GqI) can act as a specific intracellular inhibitor of Gq while not inhibiting signaling through other G protein-coupled pathways (Akhter et al, Science 280:574-577 (1998)). The GqI targets the intracellular interface between activated GPCRs and Gαq (Garrison, Hypertension Primer 2^{nd} Edition, eds Izzo and Black, American Heart Association, pgs. 63-65 (1999)). Previously, the GqI peptide has been targeted to the myocardium of transgenic mice and it has been demonstrated that in vivo in the heart, the GqI does act as a specific Gq inhibitor and prevents myocardial hypertrophy in response to pressure overload (Akhter et al, Science 280:574-577 (1998)).

In accordance with the invention, transgenic mice with VSM specific expression of the GqI have been created. Three founder lines of SM22α-GqI have been generated. The data in these mice demonstrate that these mice have significantly lower systemic blood pressure and indicate that by targeting intracellular Gq signaling, regardless of the extracellular input, a single molecule or therapy can be developed that would effectively reduce blood pressure and treat the syndrome of hypertension.

The targeting of the GqI peptide can be accomplished by linking this cDNA to a segment of the SM22α promoter, which has been previously shown to be an arterial VSM marker. However, besides marker transgenes, transgenic mice expressing a functional transgene had not previously been developed. In the SM22α-GqI mice, not only is chronic resting blood pressure significantly lower than non-transgenic littermate control (NLC) mice, arterial vessels are significantly thinner indicating that inhibiting Gq in VSM in vivo can limit cellular hypertrophy of arteries that also would benefit the hypertensive patient.

This is the first mouse model describing the VSM targeting expression of a transgene that has altered the phenotype of the animal. The experiments described herein illustrate that inhibiting signaling through Gq by expressing the GqI in VSM causes decreased chronic in vivo resting blood pressure with a concomitant decrease in arterial wall thickness and cardiac hypertrophy. This is a significant finding as these mice represent a novel model of hypotension that permit study of the role of Gq in the pathogenesis of hypertension and vascular hypertrophy. Moreover, these data, coupled with prior results, indicate that targeting intracellular Gq signaling is a novel target to lower blood pressure and to inhibit hypertrophic changes of blood vessels and the heart associated with the syndrome of hypertension.

It will be appreciated from the foregoing that the animals described herein, and cells derived therefrom, can be used in a variety of screening protocols designed to identify therapeutically effective agents. Additionally, the data derived from these animal models make it clear that anti-hypertensive compounds can be selected on the basis of their ability to target intracellular Gq signaling. Compounds identified (or identifiable) using such screens can be used in therapeutic strategies designed to lower blood pressure and inhibit other changes in blood vessels and the heart associated with hypertension. The compounds can be formulated as pharmaceutical compositions that include a pharmaceutically acceptable diluent or carrier. Such compositions can be sterile, in dosage unit form and/or suitable for administration by injection, or otherwise. Optimum dosing regimens can be readily established by one skilled in the art.

Certain aspects of the invention can be described in greater detail in the non-limiting Example that follows.

### EXAMPLE 1

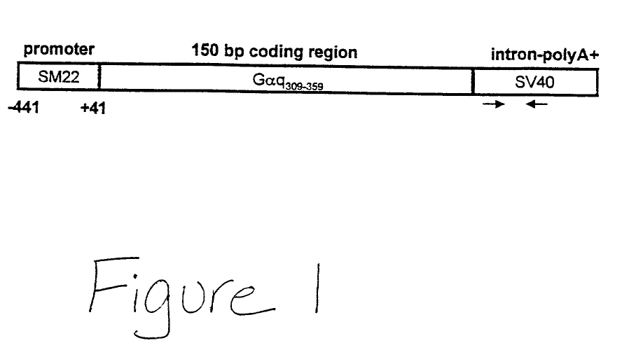

In order to understand the mechanism of GPCR signaling and specifically Gq, transgenic mice overexpressing this GRK were generated by using the SM22α promoter. To generate mice with targeted VSM expression, amplification, by standard PCR techniques, of a 481 bp portion of the SM22α promoter from mouse genomic DNA that spans from −441 to +41 (relative to transcription start) was effected. This portion of the promoter is necessary and sufficient to direct robust transcription in VSM but not in non-vascular tissues both in vivo and in vitro (Kim et al, Mol. Cell Biol. 17:2266-2278 (1997)); Solway et al, J. Biol. Chem. 270:13460-13469 (1995)). The transgene used was the coding region for muring Gαq residues 305-359 (the last 50 amino acid residues). This SM22α-GqI construct also contains a poly-adenylation signal for SV40 to provide mRNA stability and aid in detection of transgene incorporation for screening purposes by slot blot analysis of genomic DNA isolated from tail-cuts (see FIG. 1). This transgenic construct was linearized and given to the Duke University Transgenic Mouse Facility for microinjection and generation of founder lines of transgenic mice. Three founder lines for the GqI overexpressing mice have been generated preliminary studies in one of these lines of SM22α-GqI mice (GqI48) have been completed.

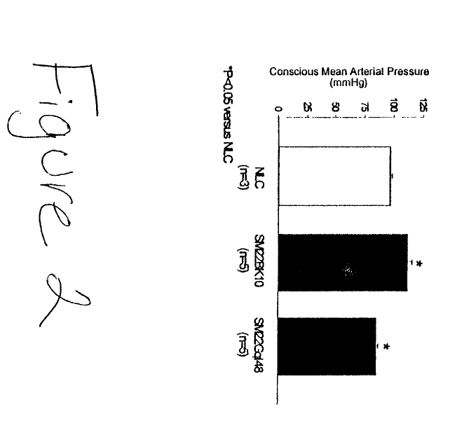

In preliminary experiments, mice expressing the peptide inhibitor of Gq signaling (GqI) in VSM (GqI48) have significantly decreased mean arterial blood pressure (MAP) (82±2 mmHg, vs. NLC 96±2 n=5, p=0.02), decreased diastolic pressure (72±2 mmHg, vs. NLC 84±3 n=5, p=0.02) and systolic pressure (GqI48: 109±3 mmHg, n=5 versus NLC: 120±3 mm g, n=3, p=0.04). FIG. 2 shows the MAP data. Thus, all three blood pressure parameters in the chronic resting state of the GqI48 mice were significantly lower than NLC mice. SM22α-GqI48 had a similar heart to body weight ratio to NLC mice demonstrating that no cardiac hypertrophy was present. However, these mice did have significantly decreased aorta wall thickness (40±2 μm vs. NLC 51±5 μM, p=0.03). These data indicate that Gq signaling plays an important role in the establishment of normal resting vascular tone and hypertrophy.

These data indicate that the SM22α promoter directs specific, robust, active vascular expression and that it is a viable promoter to use for VSM-specific expression of transgenes. Moreover, VSM targeted GqI expression leads to lower blood pressure decreased arterial wall thickness. This is a significant finding showing that Gq-mediated signaling is an important component of normal resting vascular tone and blood vessel wall thickness. Data obtained from these mice also indicate that targeting the Gq-receptor interface with the GqI is a novel strategy for inhibiting increased SVR and treating the syndrome of hypertension.

### EXAMPLE 2

Gq-Mediated Signaling is an Important Component of Normal Resting Vascular Tone and Arterial Smooth Muscle Growth

Activation of certain Gq-coupled receptors can dramatically increase mean arterial pressure (MAP), and myocardial and vascular hypertrophy. A peptide inhibitor of Gq signaling has been developed that is the carboxyl-terminus of Gaq (GqI). This region of the α-subunit interacts specifically with the activated receptor and targets inhibition of Gq coupling. To determine the role that Gq-coupled receptors play in establishing resting blood vessel tone and their contribution to hypertension, GqI was expressed specifically in vascular smooth muscle cells (SMC) of transgenic mice using a 441 bp portion of the SM22α promoter. Parameters from two distinct lines, GqI48 and GqI49, were examined in vivo and in cultured cells derived from arterial and venous vessels. Conscious mean arterial pressure (MAP) was decreased as compared to WT (GqI48 84±2 mmHg, n=5, vs WT 84±3 mmHg, n=3, p=0.02) and systolic pressure (GqI48 109±3 mmHg, n=5 vs WT 120±3 mmHg, n=3, p=0.04). Interestingly, aorta wall thickness was decreased from 51±4 μm in WT to 40±2 μm in GqI49 mice (p=0.03). To specifically address whether in vivo responses to known Gq-mediated vasoconstrictors were blocked by SMC expression of GqI, MAP was measured in response to angiotensin II (AngII) and phenylephrine (PE) in anesthetized mice. Importantly, responses were significantly attenuated tone (1 μg/kg AngII: WT 91±12%, n=9 vs GqI49 11±2% increase over resting MAP, n=2, p=0.01; 8 μg/kg PE: WT 44±11% vs GqI48 2±6%, n=5, p=0.03). Responses to isoproterenol (ISO) were unaffected (10 μg/kg ISO: WT −54±4%, n=9 vs GqI48 −53±7, n=5) demonstrating the specificity of the GqI in vivo as this Gs-coupled effect was not inhibited. These data demonstrate that Gq-mediated signaling is an important component of normal resting vascular tone and blood vessel wall thickness and that substantial angiotensin II and phenylephrine pressure responses in vivo are mediated via Gq. Targeting the Gq-receptor interface with the GqI is a novel strategy for inhibiting increased systemic vascular resistance.

### EXAMPLE 3

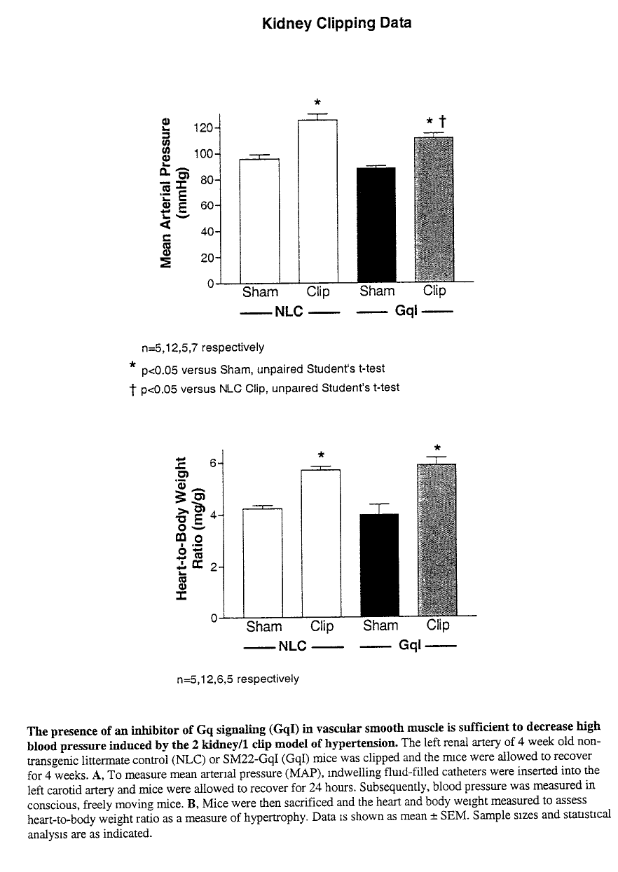

The left renal artery of 4 week old non-transgenic littermate control (NLC) or SM22-GqI (GqI) mice was clipped and the mice were allowed to recover for 4 weeks. To measure mean arterial pressure (MAP), indwellling fluid-filled catheters were inserted into the left carotid artery and mice were allowed to recover for 24 hours. Subsequently, blood pressure was measured in conscious, freely moving mice. Mice were then sacrificed and the heart and body weights measured to assess heart-to-body weight ratio as a measure of hypertrophy. The results indicate that mice with the GqI have less hypertension (blood pressure increase) indicating that this is a means of lowering blood pressure. Data in FIGS. 3A and 3B are shown as mean +/−SEM; sample sizes and statistical analysis are as indicated.

All documents cited above are hereby incorporated in their entirety by reference.

In the claims:

1. A non-human transgenic animal that expresses GqI specifically in vascular smooth muscle cells thereof, and cells derived from said animal.

2. The animal according to claim 1 wherein said animal is a rodent.

3. The animal according to claim 2 wherein said animal is a mouse.

4. A method of screening a test compound for anti-hypertensive activity comprising determining the ability of said compound to inhibit intracellular Gq signaling in vascular smooth muscle cells.

5. A compound identifiable according to the method of claim 4.

6. A method of treating hypertension in a mammal comprising administering a compound identifiable by the method according to claim 4 in an amount sufficient to effect said treatment.
